(19) United States
(12) Patent Application Publication
(10) Pub. No.: US 2020/0289249 A1
(43) Pub. Date: Sep. 17, 2020

(54) USAGE OF MELT SPUN POLYOLEFIN FINE FIBERS FOR SKIN REGENERATION AND MESH IMPLANTATION

(71) Applicant: The Board of Regents of The University of Texas system, Austin, TX (US)

(72) Inventors: Karen Lozano, Edinburg, TX (US); Robert Gilkerson, Austin, TX (US); Karen Lorena Baylon Quinones, Monterry (MX); Alex Elias Zuniga, Monterrey (MX)

(21) Appl. No.: 16/082,010

(22) PCT Filed: Mar. 3, 2017
(86) PCT No.: PCT/US2017/020774

§ 371 Date: Sep. 4, 2018

Related U.S. Application Data
(60) Provisional application No. 62/303,202, filed on Mar. 3, 2016.

(51) Int. Cl. A61F 2/00; D01F 6/06; D01F 1/10

(52) U.S. Cl. CPC A61F 2/0063; A61K 9/0092; D01F 1/103; D01F 6/06

(57) ABSTRACT

Described herein is the application of centrifugal spinning to provide a polypropylene (PP) nanofiber environment for cell growth. PP nanofiber mats, formed by centrifugal spinning are treated with one or more antimicrobial agents. The treated PP material was shown to have a 10 reduced rate of rejection and promote cell growth and proliferation.

### STATEMENT REGARDING FEDERALLY SPONSORED RESEARCH OR DEVELOPMENT

This work was supported by National Science Foundation under DMR grant #0934157 (PREM- and DMR grant #1040419 (MRI: Acquisition of an ESEM). The government has certain rights in the invention.

### BACKGROUND OF THE INVENTION

### 1. Field of the Invention

The present invention generally relates to the field of polyolefin fiber production. More specifically, the invention relates to the formation and use of polyolefin fibers for medical uses.

### 2. Description of the Relevant Art

Fibers having small diameters (e.g., micrometer (“micron”) to nanometer (“nano”)) are useful in a variety of fields from the clothing industry to military applications. For example, in the biomedical field, there is a strong interest in developing structures based on nanofibers that provide a scaffolding for tissue growth to effectively support living cells.

Hernia repair is one of the most common surgeries performed globally. The number of procedures has been increasing and is predicted to further increase due to several risk factors such as obesity and abdominal surgeries. The use of hernia mesh products to surgically repair or reconstruct anatomical defects has been widely adopted. The surgical mesh firmly strengthens the weakened area and provides tension-free repair that facilitates the incorporation of fibrocollagenous tissue. However, there are many types of meshes and there is a strong controversy regarding optimum performance and success of surgical procedures. Researchers have investigated metals, composites, polymers and biodegradable biomaterials in their quest to attain the ideal surgical mesh and implantation procedure. The sought-after characteristics are: inertness, resistance to infection, the ability to maintain adequate long-term tensile strength to prevent early recurrence, rapid incorporation into the host tissue, adequate flexibility to avoid fragmentation, non-carcinogenic response and the capability to restore the natural respiratory movements of the abdominal wall.

Currently, utilized surgical meshes exhibit many but not all of these desired characteristics. Therefore, current research efforts focus on providing potential solutions that range from the utilization of novel materials to new designs that could ameliorate current shortcomings.

Centrifugal spinning (e.g., Forcespinning) is a newly developed method that uses centrifugal forces to drive the material through a designed set of orifices within a spinneret, has become an attractive method for polymer melt and solution spinning. Centrifugal spinning also provides the advantage of producing nanofibers from materials with a low dielectric constant which had not been previously produced (in its pure state) by electrospinning.

Other materials had been proven to have difficulties such as polypropylene where appropriate secondary solvents have to be selected because of low dielectric constant of the primary solvent or salts need to be added to enhance conductivity of the solution in order to be electrospun. In the case of centrifugal spinning, this is not a factor to be considered and nanofibers have been successfully obtained either in solution or melt process without the need to either potentially contaminate the sample with the salts or with the addition of more chemicals adding cost to the produced fibers.

### SUMMARY OF THE INVENTION

In an embodiment, fibers for medical use are composed of polypropylene and an antimicrobial agent. For medical use it is preferred that the fibers have an average diameter of less than about 500 nm.

In an embodiment, the polypropylene polymer has a melt flow rate of between about 30 g/10 min to about 1600 g/10 min. In an embodiment, the polypropylene polymer has a glass transition temperature of below about 250° C.

The antimicrobial agent may be an antibiotic agent or an antifungal agent. Specific examples of antimicrobial agents include, but are not limited to, tannic acid, chitosan, penicillin, streptomycin antibiotics, cefazolin, erythromycin, cefoxitin, cefotetan, and/or fungizone antimycotic.

In an embodiment, a method of producing the polypropylene fibers includes placing a mixture comprising polypropylene polymer and an antimicrobial agent into a body of a fiber producing device, the body comprising one or more openings. The polypropylene polymer is heated to a temperature above the glass transition temperature of the polypropylene polymer. The fiber producing device is rotated at a speed of at least about 500 rpm. The rotation of the fiber producing device causes the heated mixture of the polypropylene polymer and the antimicrobial agent in the body to be passed through one or more openings to produce polypropylene microfibers and/or polypropylene nanofibers comprising the polypropylene polymer and the antimicrobial agent. The produced antimicrobial polypropylene microfibers and/or antimicrobial polypropylene nanofibers are collected and may be used for the formation of medical meshes.

In an embodiment, a method of producing the polypropylene fibers includes placing a polypropylene polymer into a body of a fiber producing device, the body comprising one or more openings. The polypropylene polymer is heated to a temperature above the glass transition temperature of the polypropylene polymer. The fiber producing device is rotated at a speed of at least about 500 rpm, wherein rotation of the fiber producing device causes the heated polypropylene polymer in the body to be passed through one or more openings to produce polypropylene microfibers and/or polypropylene nanofibers. At least a portion of the produced polypropylene microfibers and/or polypropylene nanofibers are collected and treated with an antimicrobial agent. In one embodiment, treating the polypropylene microfibers and/or polypropylene nanofibers includes dipping the fibers into a solution of the antimicrobial agent. In another embodiment, treating the polypropylene microfibers and/or polypropylene nanofibers includes spraying the fibers with a solution of the antimicrobial agent.

In either method, the polypropylene microfibers and/or polypropylene nanofibers are created without subjecting the polypropylene microfibers and/or polypropylene nanofibers, during their creation, to an externally applied electric field.

In one embodiment, the polypropylene microfibers and/or polypropylene nanofibers are collected as a mat of the polypropylene microfibers and/or polypropylene nanofibers. In another embodiment, the polypropylene microfibers and/or polypropylene nanofibers are collected by depositing the polypropylene microfibers and/or polypropylene nanofibers onto a support.

In one embodiment, a polypropylene medical mesh comprises polypropylene microfibers and/or polypropylene nanofibers and an antimicrobial agent formed by the method described above. The polypropylene medical mesh, in some embodiments, includes a support coated with the polypropylene microfibers and/or polypropylene nanofibers comprising an antimicrobial agent. In an embodiment, polypropylene medical mesh has a biaxial tensile strength of at least about 20 kPa.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1A shows a top view of a fiber producing device and a collection wall;

FIG. 1B shows a projection view of a fiber producing device that includes a fiber producing device as depicted in FIG. 1A and a collection wall;

FIG. 2A shows a partially cut-away perspective view of an embodiment of a fiber producing system;

FIG. 2B depicts a cross-sectional view of a fiber producing system;

FIG. 3 depicts a schematic diagram of fiber formation using centrifugal spinning;

FIG. 4 depicts the schematic diagram of a heated fiber producing system;

FIG. 5 depicts a schematic diagram of a continuous fiber coating system;

FIG. 6 depicts a picture of polypropylene fibers obtained by a melt process;

FIG. 7 shows the fiber diameter distribution for PP fibers for MFR of 36 g/10 min;

FIG. 8 shows the fiber diameter distribution for PP fibers for MFR of 500 g/10 min;

FIG. 9 shows the fiber diameter distribution for PP fibers for MFR of 1200 g/10 min;

FIG. 10 shows the fiber diameter distribution for PP fibers for, MFR of 1550 g/10 min;

FIGS. 11 and 12 depict scanning electron micrographs of PP fibers;

FIG. 13 depicts PP fibers obtained as free standing non-woven mats;

FIG. 14 depicts PP fibers deposited onto a substrate;

FIG. 15 depicts a melt spun PP self-standing continuous nanofibers mat;

FIG. 16 depicts the XRD curves for PP in powder form and developed nanofibers;

FIG. 17 depicts a DSC plot of melt spun PP fibers;

FIG. 18 depicts TGA curves for melt spun PP fibers;

FIG. 19 depicts culture testing of coated and uncoated PP mesh; and

FIG. 20 depicts cell proliferation studies on PP nanofibers versus standard surgical mesh.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a method or apparatus that “comprises,” “has,” “includes” or “contains” one or more steps or elements possesses those one or more steps or elements, but is not limited to possessing only those one or more steps or elements. Likewise, an element of an apparatus that “comprises,” “has,” “includes” or “contains” one or more features possesses those one or more features, but is not limited to possessing only those one or more features.

In an embodiment, a method of producing polypropylene fibers for medical use includes forming polypropylene microfibers and/or nanofibers using a melt spinning process. In a melt spinning process, the polypropylene polymer is placed into a body of a fiber producing device. The body of the fiber producing device comprises one or more openings. The openings are sized such that when the material disposed in the body is ejected, the material will be formed into microfibers and/or nanofibers. As used herein the term “microfibers” refers to fibers having a diameter of less than 1 mm. As used herein the term “nanofibers” refers to fibers having a diameter of less than 1 μm.

The polypropylene polymer is heated to form a melt that is capable of being ejected from the body. Generally, polypropylene pellets are heated to a temperature above the melting temperature to form a melt. The polypropylene pellets/powder may be melted prior to placing the polypropylene polymer in the body of the fiber producing device and/or while the polypropylene is in the body of the fiber producing device. Suitable polypropylene polymers include, but are not limited to, polypropylene polymers that have a melt flow rate of between about 30 g/10 min to about 1600 g/10 min. Additionally, suitable polypropylene polymers include, but are not limited to, polypropylene polymers having a glass transition temperature of below about 250° C.

The fiber producing device is rotated at a speed of at least about 500 rpm. Rotation of the fiber producing device causes the heated polypropylene polymer in the body to be passed through one or more openings to produce polypropylene microfibers and/or polypropylene nanofibers. The polypropylene microfibers and/or polypropylene nanofibers are created without subjecting the polypropylene microfibers and/or polypropylene nanofibers, during their creation, to an externally applied electric field (known, hereinafter, as “centrifugal spinning”).

Apparatuses and methods that may be used to create the polypropylene microfibers and/or polypropylene nanofibers are described in the following U.S. Published Patent Applications: 2009/0280325; 2009/0269429; 2009/0232920; and 2009/0280207, all of which are incorporated herein by reference.

The produced polypropylene microfibers and/or polypropylene nanofibers are collected and treated with an antimicrobial agent. In some embodiments, the polypropylene microfibers and/or polypropylene nanofibers are collected as a mat of the polypropylene microfibers and/or polypropylene nanofibers. In other embodiments, the polypropylene microfibers and/or polypropylene nanofibers are collected by depositing the polypropylene microfibers and/or polypropylene nanofibers onto a support. Suitable supports include, but are not limited to polypropylene surgical mesh. Mesh formed from other materials may also be used as a support.

An antimicrobial is an agent that kills microorganisms or inhibits their growth. Antimicrobial agents include antibacterials and antifungals. Exemplary antimicrobial compounds include, but are not limited to, tannic acid, chitosan, penicillin, streptomycin antibiotics, cefazolin, erythromycin, cefoxitin, cefotetan, and/or fungizone antimycotic. The polypropylene microfibers and/or polypropylene nanofibers may be treated by dipping the fibers into a solution of the antimicrobial agent. Alternatively, the polypropylene microfibers and/or polypropylene nanofibers may be treated by spraying the fibers with a solution of the antimicrobial agent.

In an alternate embodiment, antimicrobial polypropylene fibers are produced by melt spinning of a mixture of polypropylene and an antimicrobial agent. In one method, a mixture that includes polypropylene polymer and an antimicrobial agent is placed into a body of a fiber producing device. The body of the fiber producing device includes one or more openings. The polypropylene polymer is heated to a temperature above the glass transition temperature of the polypropylene polymer. The polymer/antimicrobial mixture may be heated prior to placing the polymer in the body, or the mixture may be placed in the body and heated by heating the body. Once the polymer/antimicrobial mixture is heated, the fiber producing device is rotated at a speed of at least about 500 rpm. Rotation of the fiber producing device causes the heated mixture of the polypropylene polymer and the antimicrobial agent in the body to be passed through one or more openings to produce polypropylene microfibers and/or polypropylene nanofibers comprising the polypropylene polymer and the antimicrobial agent.

Nanofibrous structures present several advantages as scaffolds for tissue engineering, such as high specific surface area for cell attachment, higher microporous structure and a 3D micro environment for cell-cell and cell-biomaterial contact. These structures, when compared with commercial surgical meshes, possess higher porosity and smaller pore size. These properties make nanofiber systems suitable for biomaterials used in wound care, drug delivery, and scaffolds for tissue regeneration.

Scaffolds for tissue engineering must possess a porous structure that can facilitate cell migration, a balance between surface hydrophilicity and hydrophobicity for cell attachment, mechanical properties comparable to natural tissue, and biocompatibility. Studies have shown that the above mentioned characteristics are also highly influenced by average diameter of the fibers and pore size. Effective cell attachment and proliferation has been observed in fiber systems with average diameters smaller than 1 μm and average pore size of 14 μm. In commercially available meshes, even when it has been shown that cells are able to proliferate in micrometer/macrometer regimes, the cells in fact have difficulty attaching and proliferating. Cells are seen around the fibers whereas on nanofiber based meshes, the cells attach to the fibers and quickly proliferate while making strong contact with underlying nanofibers, therefore promoting interlayer growth.

The application of nanofiber systems has been hampered due to its poor mechanical properties and nanofiber availability. Most of the available studies have focused on nanofibers prepared through solution processes. The properties of the developed fibers can be controlled by different parameters such as utilized solvent, concentration of polymer, processing methods, and ambient conditions. For example, in the case of nanofibers made of polypropylene (one of the highly used polymers for commercially available surgical meshes), decahydronaphthalene (decalin) and cyclohexane have been used as preferred solvents. Polypropylene nanofibers prepared with cyclohexane exhibited a rougher surface when compared to the fibers prepared with decalin, suggesting that the surface morphology of the nanofibers depend on the boiling point of each solvent. When stress-strain behaviors of the nanofibers are investigated, a tensile strength of 61.4±1.5 MPa with 35.2±1.7% of strain, and a Young modulus of 174.6±1.7 MPa was obtained for the decalin based nanofibers, whilst the cyclohexane nanofibers exhibit a tensile strength of 18.2±1.1 MPa with 46.7±1.2% of elongation and a Young modulus of 39.1±1.4 MPa. The above mentioned results were obtained from bundles of nanofibers rather than individual fibers, these properties are strongly dependent on fiber orientation within the tested sample, bonding between fibers, and slip of one fiber over another.

Regarding nanofiber availability, there are several methods to prepare nanofiber systems. These methods include wet chemistry, Electrospinning (ES) and Forcespinning® (FS) techniques. Most of the available literature has used ES processes, however, ES processes have been limited to laboratory-based research given the challenges associated with increasing yield and opportunity to work with melt based systems. FS, a technique that has been recently introduced is based on developing nanofibers through the application of centrifugal forces. The method has been proven effective to produce yields that could satisfy industry requirements (e.g., several hundred meters per minute) as well as to produce nanofibers from melt based systems therefore removing the requirement of a solvent and subsequently the potential contamination of the materials with toxic organic solvents, and cost associated with the solvent itself and solvent recovery procedures.

Polypropylene surgical mesh has been utilized extensively for wound healing applications due to its tensile strength properties. Despite this, polypropylene surgical mesh shows a high rate of rejection by the body, approaching 40% of patients implanted. Unexpectedly, when the same polypropylene polymer is spun (in the absence of solvents) into microfiber/nanofiber-based membranes using centrifugal spinning, the produced fiber supports much more robust cell proliferation and growth than conventional polypropylene surgical mesh. Standard polypropylene surgical mesh does not provide a spatial environment that permits the proliferation of mammalian fibroblast cells. However, it was found that the same polypropylene polymer, centrifugally spun, from a melt, into nanofiber membranes, very effectively promotes cell proliferation and viability.

In one embodiment, a polypropylene medical mesh is produced comprising polypropylene microfibers and/or polypropylene nanofibers and an antimicrobial agent. In one embodiment, the polypropylene medical mesh is formed as a mat using one or more of the polypropylene/antimicrobial fiber producing processes described herein. Alternatively, one or more of the polypropylene/antimicrobial fiber producing processes described herein may be deposited onto a biocompatible support.

One purpose of polypropylene surgical mesh is to promote cell proliferation. Polypropylene microfibers and/or polypropylene nanofibers material that have been treated with antimicrobial agents may be used as scaffold in tissue engineering, for different applications such as wound dressing, hernia repair, pelvic organ prolapse support, cardiac patches and as a coating for different implantable medical devices that need to be integrated within the body.

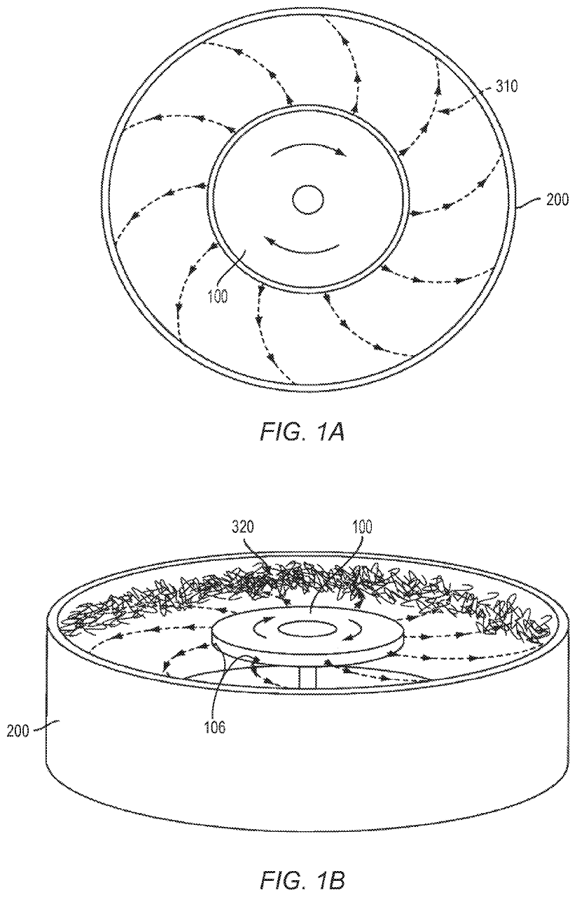

FIG. 1A shows a top view of an exemplary fiber producing system that includes a fiber producing device 100 and a collection wall 200. FIG. 1B shows a projection view of a fiber producing system that includes a fiber producing device 100 and a collection wall 200. As depicted, fiber producing device 100 is spinning clockwise about a spin axis, and material is exiting openings 106 of the fiber producing device as fibers 320 along various pathways 310. The fibers are being collected on the interior of the surrounding collection wall 200.

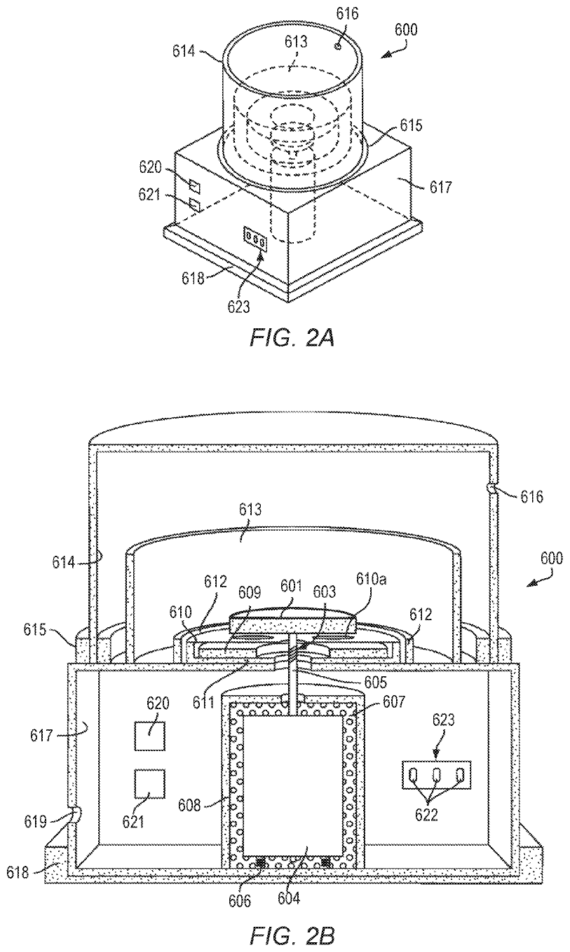

FIG. 2A shows a partially cut-away perspective view of an embodiment of a fiber producing system 600. FIG. 2B depicts a cross-sectional view of fiber producing system 600. System 600 includes fiber producing device 601, which has peripheral openings and is coupled to a threaded joint 603, such as a universal threaded joint, which, in turn, is coupled to a motor 604 via a shaft 605. Motor 604, such as a variable speed motor, is supported by support springs 606 and is surrounded by vibration insulation 607 (e.g., high-frequency vibration insulation). A motor housing 608 encases the motor 604, support springs 606 and vibration insulation 607. A heating unit 609 is enclosed within enclosure 610 (e.g., a heat reflector wall) that has openings 610a that direct heat (thermal energy) to fiber producing device 601. In the embodiment shown, heating unit 609 is disposed on thermal insulation 611. Surrounding the enclosure 610 is a collection wall 612, which, in turn, is surrounded by an intermediate wall 613. A housing 614 seated upon a seal 615 encases fiber producing device 601, heating enclosure 610, collection wall 612 and intermediate wall 613. An opening 616 in the housing 614 allows for introduction of fluids (e.g., gases such as air, nitrogen, helium, argon, etc.) into the internal environment of the apparatus, or allows fluids to be pumped out of the internal environment of the apparatus. The lower half of the system is encased by a wall 617 which is supported by a base 618. An opening 619 in the wall 617 allows for further control of the conditions of the internal environment of the apparatus. Indicators for power 620 and electronics 621 are positioned on the exterior of the wall 617 as are control switches 622 and a control box 623.

A control system of an apparatus 622 allows a user to change certain parameters (e.g., RPM, temperature, and environment) to influence fiber properties. One parameter may be changed while other parameters are held constant, if desired. One or more control boxes in an apparatus may provide various controls for these parameters, or certain parameters may be controlled via other means (e.g., manual opening of a valve attached to a housing to allow a gas to pass through the housing and into the environment of an apparatus). It should be noted that the control system may be integral to the apparatus (as shown in FIGS. 2A and 2B) or may be separate from the apparatus. For example, a control system may be modular with suitable electrical connections to the apparatus.

In certain methods described herein, material spun in a fiber producing device may undergo varying strain rates, where the material is kept as a melt or solution. Since the strain rate alters the mechanical stretching of the fibers created, final fiber dimension and morphology may be significantly altered by the strain rate applied. Strain rates are affected by, for example, the shape, size, type and RPM of a fiber producing device. Altering the viscosity of the material, such as by increasing or decreasing its temperature or adding additives (e.g., thinner), may also impact strain rate. Strain rates may be controlled by a variable speed fiber producing device. Strain rates applied to a material may be varied by, for example, as much as 50-fold (e.g., 500 RPM to 25,000 RPM).

Temperatures of the material, fiber producing device and the environment may be independently controlled using a control system. The temperature value or range of temperatures employed typically depends on the intended application. For example, for many applications, temperatures of the material, fiber producing device and the environment typically range from −4° C. to 400° C. Temperatures may range as low as, for example, −20° C. to as high as, for example, 2500 C. For solution spinning, ambient temperatures of the fiber producing device are typically used.

As the material is ejected from the spinning fiber producing device, thin jets of the material are simultaneously stretched and dried in the surrounding environment. Interactions between the material and the environment at a high strain rate (due to stretching) lead to solidification of the material into polymeric fibers, which may be accompanied by evaporation of solvent. By manipulating the temperature and strain rate, the viscosity of the material may be controlled to manipulate the size and morphology of the polymeric fibers that are created. With appropriate manipulation of the environment and process, it is possible to form polymeric fibers of various configurations, such as continuous, discontinuous, mat, random fibers, unidirectional fibers, woven and unwoven, as well as fiber shapes, such as circular, elliptical and rectangular (e.g., ribbon). Other shapes are also possible. The produced fibers may be single lumen or multi-lumen.

By controlling the process parameters, fibers can be made in micron, sub-micron and nano-sizes, and combinations thereof. In general, the fibers created will have a relatively narrow distribution of fiber diameters. Some variation in diameter and cross-sectional configuration may occur along the length of individual fibers and between fibers.

Generally speaking, a fiber producing device helps control various properties of the fibers, such as the cross-sectional shape and diameter size of the fibers. More particularly, the speed and temperature of a fiber producing device, as well as the cross-sectional shape, diameter size and angle of the outlets in a fiber producing device, all may help control the cross-sectional shape and diameter size of the fibers. Lengths of fibers produced may also be influenced by fiber producing device choice.

The speed at which a fiber producing device is spun may also influence fiber properties. The speed of the fiber producing device may be fixed while the fiber producing device is spinning, or may be adjusted while the fiber producing device is spinning. Those fiber producing devices whose speed may be adjusted may, in certain embodiments, be characterized as “variable speed fiber producing devices.” In the methods described herein, the structure that holds the material may be spun at a speed of about 500 RPM to about 25,000 RPM, or any range derivable therein. In certain embodiments, the structure that holds the material is spun at a speed of no more than about 50,000 RPM, about 45,000 RPM, about 40,000 RPM, about 35,000 RPM, about 30,000 RPM, about 25,000 RPM, about 20,000 RPM, about 15,000 RPM, about 10,000 RPM, about 5,000 RPM, or about 1,000 RPM. In certain embodiments, the structure that holds the material is rotated at a rate of about 5,000 RPM to about 25,000 RPM.

In an embodiment, material may be positioned in a reservoir of the fiber producing device. The reservoir may, for example, be defined by a concave cavity of the fiber producing device. In certain embodiments, the fiber producing device includes one or more openings in communication with the concave cavity. The fibers are extruded through the opening while the fiber producing device is rotated about a spin axis. The one or more openings have an opening axis that is not parallel with the spin axis. The fiber producing device may include a body that includes the concave cavity and a lid positioned above the body such that a gap exists between the lid and the body, and the nanofiber is created as a result of the rotated material exiting the concave cavity through the gap.

Certain fiber producing devices have openings through which material is ejected during spinning. Such openings may take on a variety of shapes (e.g., circular, elliptical, rectangular, square, triangular, or the like) and sizes: (e.g., diameter sizes of 0.01-0.80 mm are typical). The angle of the opening may be varied between ±15 degrees. The openings may be threaded. An opening, such as a threaded opening, may hold a needle, where the needle may be of various shapes, lengths and gauge sizes. Threaded holes may also be used to secure a lid over a cavity in the body of a fiber producing device. The lid may be positioned above the body such that a gap exists between the lid and the body, and a fiber is created as a result of the spun material exiting the cavity through the gap. Fiber producing devices may also be configured such that one fiber producing device may replace another within the same apparatus without the need for any adjustment in this regard. A universal threaded joint attached to various fiber producing devices may facilitate this replacement. Fiber producing devices may also be configured to operate in a continuous manner.

Any method described herein may further comprise collecting at least some of the microfibers and/or nanofibers that are created. As used herein “collecting” of fibers refers to fibers coming to rest against a fiber collection device. After the fibers are collected, the fibers may be removed from a fiber collection device by a human or robot. A variety of methods and fiber (e.g., nanofiber) collection devices may be used to collect fibers. For example, regarding nanofibers, a collection wall may be employed that collects at least some of the nanofibers. In certain embodiments, a collection rod collects at least some of the nanofibers. The collection rod may be stationary during collection, or the collection rod may be rotated during collection. Regarding the fibers that are collected, in certain embodiments, at least some of the fibers that are collected are continuous, discontinuous, mat, woven, unwoven or a mixture of these configurations. In some embodiments, the fibers are not bundled into a cone shape after their creation. In some embodiments, the fibers are not bundled into a cone shape during their creation. In particular embodiments, fibers are not shaped into a particular configuration, such as a cone figuration, using air, such as ambient air, that is blown onto the fibers as they are created and/or after they are created.

Present method may further comprise, for example, introducing a gas through an inlet in a housing, where the housing surrounds at least the fiber producing device. The gas may be, for example, nitrogen, helium, argon, or oxygen. A mixture of gases may be employed, in certain embodiments.

The environment in which the fibers are created may comprise a variety of conditions. For example, any fiber discussed herein may be created in a sterile environment. As used herein, the term “sterile environment” refers to an environment where greater than 99% of living germs and/or microorganisms have been removed. In certain embodiments, “sterile environment” refers to an environment substantially free of living germs and/or microorganisms. The fiber may be created, for example, in a vacuum. For example the pressure inside a fiber producing system may be less than ambient pressure. In some embodiments, the pressure inside a fiber producing system may range from about 1 millimeters (mm) of mercury (Hg) to about 700 mm Hg. In other embodiments, the pressure inside a fiber producing system may be at or about ambient pressure. In other embodiments, the pressure inside a fiber producing system may be greater than ambient pressure. For example the pressure inside a fiber producing system may range from about 800 mm Hg to about 4 atmospheres (atm) of pressure, or any range derivable therein.

In certain embodiments, the fiber is created in an environment of 0-100% humidity, or any range derivable therein. The temperature of the environment in which the fiber is created may vary widely. In certain embodiments, the temperature of the environment in which the fiber is created can be adjusted before operation (e.g., before rotating) using a heat source and/or a cooling source. Moreover, the temperature of the environment in which the fiber is created may be adjusted during operation using a heat source and/or a cooling source. The temperature of the environment may be set at sub-freezing temperatures, such as −20° C., or lower. The temperature of the environment may be as high as, for example, 2500° C.

The fibers that are created may be, for example, one micron or longer in length. For example, created fibers may be of lengths that range from about 1 μm to about 50 cm, from about 100 μm to about 10 cm, or from about 1 mm to about 1 cm. In some embodiments, the fibers may have a narrow length distribution. For example, the length of the fibers may be between about 1 μm to about 9 μm, between about 1 mm to about 9 mm, or between about 1 cm to about 9 cm. In some embodiments, when continuous methods are performed, fibers of up to about 10 meters, up to about 5 meters, or up to about 1 meter in length may be formed. In certain embodiments, the cross-section of the fiber may be circular, elliptical or rectangular. Other shapes are also possible. The fiber may be a single-lumen lumen fiber or a multi-lumen fiber.

In another embodiment of a method of creating a fiber, the method includes: spinning material to create the fiber; where, as the fiber is being created, the fiber is not subjected to an externally-applied electric field or an externally-applied gas; and the fiber does not fall into a liquid after being created.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Experimental Method

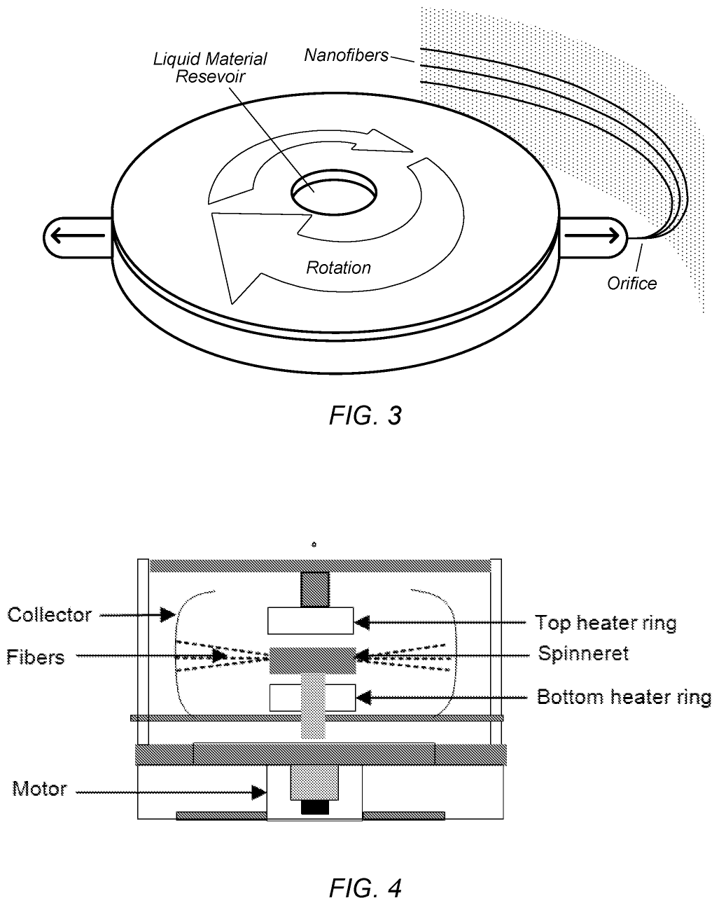

FIG. 3 shows a schematic diagram of fiber formation using centrifugal spinning. The polymer solution or melt are forced through the orifices of the spinneret by applying centrifugal force. As polymer solution or melt is ejected through the orifices, continuous polymer jets are formed and are stretched into formation of fine web of fibers due to applied centrifugal force and shear force acting across the tip of orifices of the spinneret.

The web is collected on a custom designed collector system. Fiber formation and morphology of the formed web are dictated by solution concentration (in case of solution spinning), melt viscosity (for melt spinning), rotational speed, distance between collection system and spinneret and gauge size of the spinneret. Centrifugal spinning of polypropylene (PP) melt was carried out on the lab scale equipment and FIG. 4 shows the schematic diagram of a heated fiber producing system. The polymer was loaded onto the spinneret and was melted by engaging both upper and bottom heater rings. Polypropylene of different melt flow rate (MFR) was studied: Exxon Mobil Polypropylene Homopolymer 3155 (MFR of 36 g/10 min); Lyondell basell Metocene MF 650 W (MFR of 500 g/10 min); Lyondell basell Metocene MF 650 X (MFR of 1200 g/10 min); and ExxonMobil Achieve grade 69361G (MFR of 1550 g/10 min). Polypropylene material was melted at 225° C. inside the spinneret and rotational speeds varied from 8,000 to 12,000 rpm for 30 seconds.

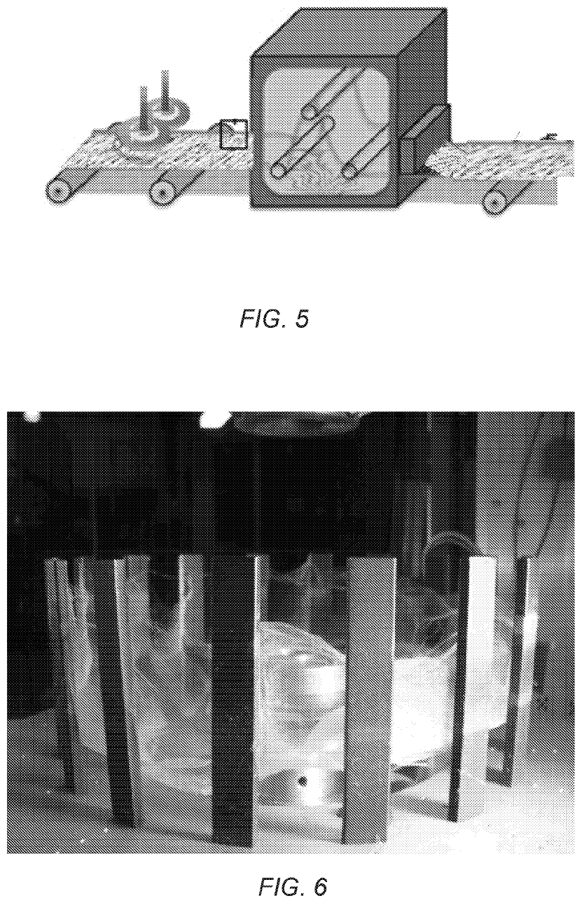

FIG. 5 depicts a system for preparing a mesh, coated with melt spun polypropylene fibers which includes a coating station for applying the antimicrobial agents to the fibers/mesh. The system 500 includes one or more fiber producing devices 510. Fibers produced by the fiber producing devices 510 are deposited onto a substrate (e.g., a mesh substrate) that is passed under the fiber producing devices. The substrate is moved past the fiber producing device in a batch or continuous fashion. In the embodiment depicted in FIG. 5, the substrate is attached to a conveyor system 520, which is used to transport and collect the substrate during use. The coated substrate is transferred from the fiber producing devices into coating station 530. In the dipping station, the fibers and/or the substrate are coated with an antimicrobial agent. The antimicrobial agent, in one embodiment, is applied as a solution to the fibers/substrate. In one embodiment, the substrate passes through a solution of the antimicrobial agents in an appropriate solvent. Alternatively, a solution of the antimicrobial agent may be sprayed into the fibers/substrate. The coating station may include a heating system to accelerate the removal of the solvent used to solubilize the antimicrobial agent.

A fiber web was collected on a glass slide for scanning electron microscopy (SEM) analysis (Hitachi S-4300 SE/N). Samples were gold sputtered. To assess fiber diameter at least 500 nanofibers randomly selected from 20 SEM micrographs were used. Fiber diameter measurement was conducted using Image J software. For the fiber diameter results presented here the measurements were made in triplicate. All replicates were performed at similar operating conditions and process parameters. The crystallinity analysis was performed using a Differential Scanning calorimeter Q series instrument (DSC Q100) calibrated with indium standard under nitrogen atmosphere (50 ml/min). The samples were first heated to 250° C. at a rate of 5° C./min, held isothermal for 15 minutes at 250° C. and cooled to room temperature (a second cycle was run under same conditions). X-Ray Diffraction data was collected in transmission mode on a Rigaku R-Axis Spider diffractometer with an image plate detector using a graphite monochromator with Cu Ka radiation (λ=1.5418 Å). The fiber samples were mounted on a Hampton Research CryoLoop. Typical acquisition times were 15 minutes. Thermal stability and weight loss studies were performed on a TA Instruments TGA Q500. The samples were analyzed from 20° C. to 700° C. at a heating rate of 10° C./min. All the analyses were performed under a N₂ atmosphere with a flow rate of 40 ml/min.

### Results and Discussions

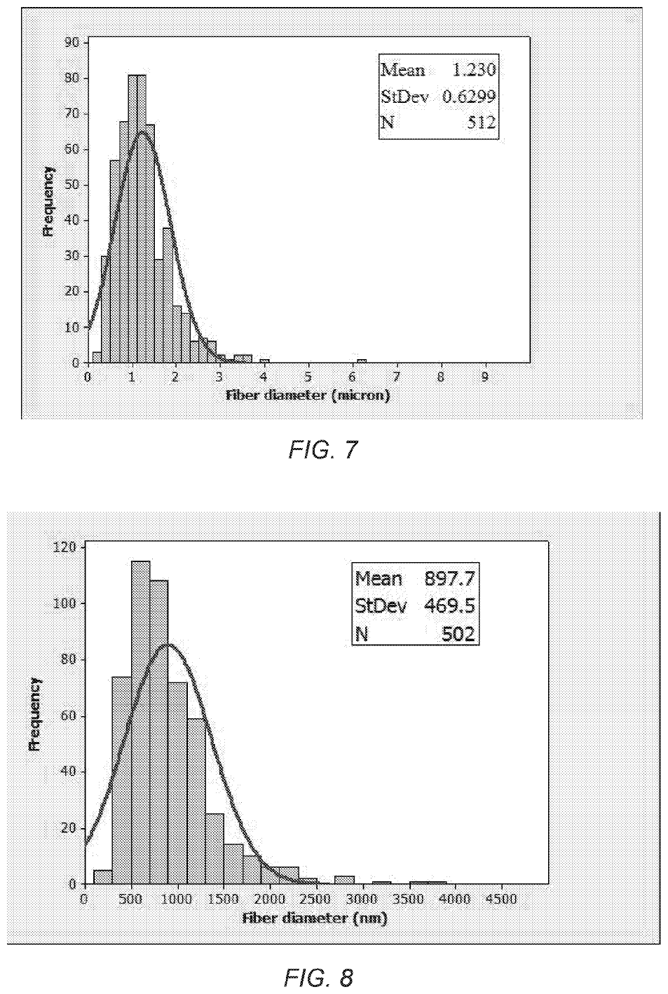

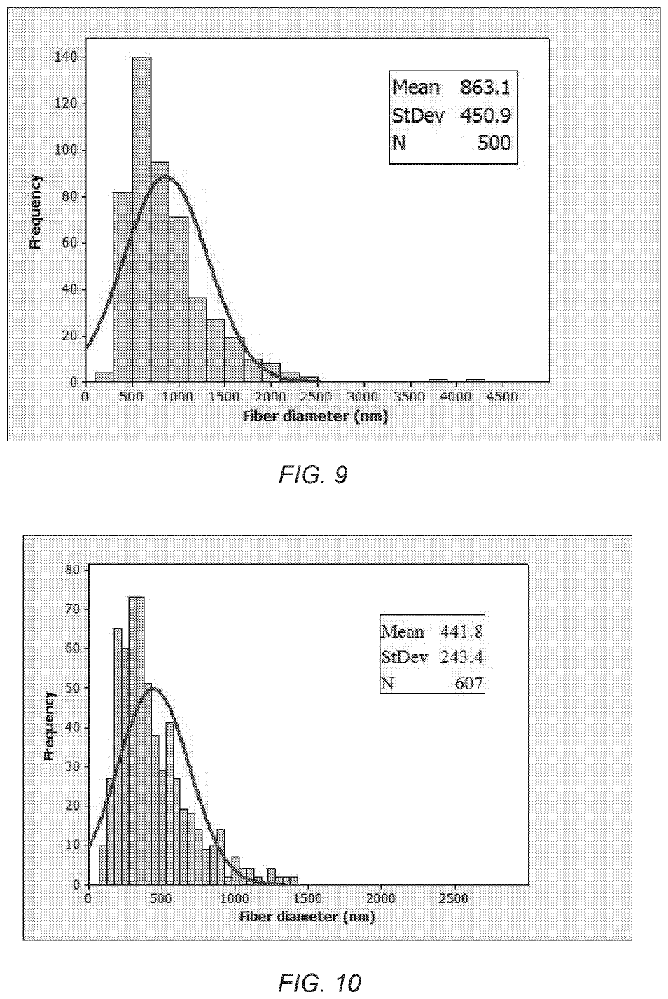

Polypropylene fibers were obtained as seen in FIG. 6. Lower rotational speeds resulted in larger fiber diameter. Polypropylene with varying MFR from 36 to 1550 g/10 min were studied for the same process conditions of 12,000 rpm and melted at 225° C. without altering chamber environmental conditions. Therefore, the fibers experienced restricted extensional flow given the rapid cooling after exiting the spinneret. The fibers were continuous, long and uniform fiber diameter was observed through the length of the fibers. FIGS. 7-10 show the fiber diameter distribution for PP fibers for MFR of 36 (FIG. 7), MFR of 500 (FIG. 8), MFR of 1200 (FIG. 9), and MFR of 1550 g/10 min (FIG. 10) and indicates that the average fiber diameter decreases with the increased MFR and standard deviations also reduced with increased MFR.

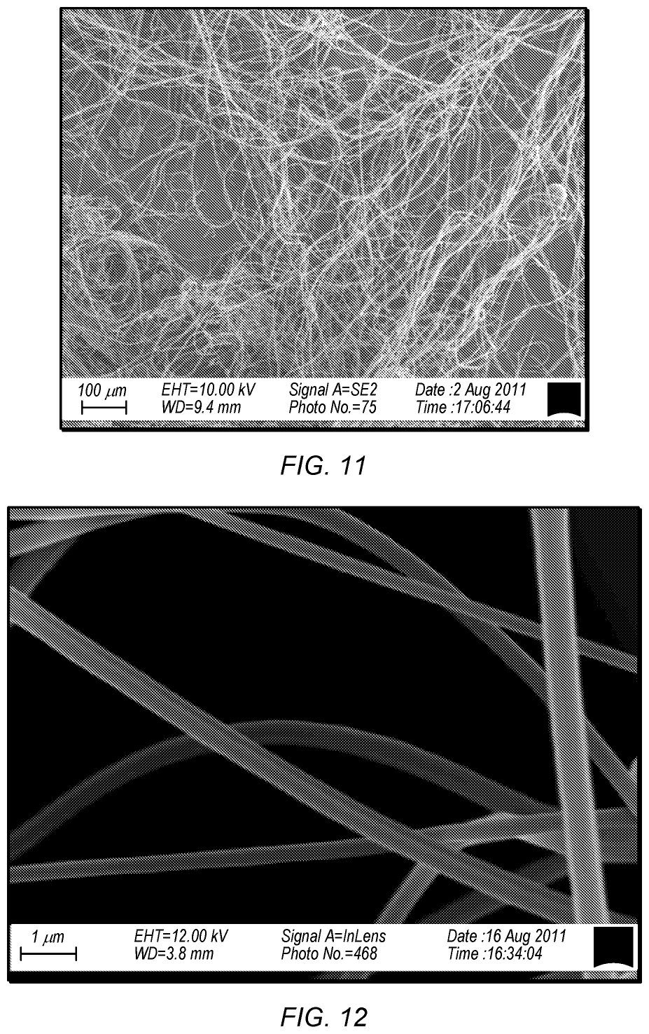

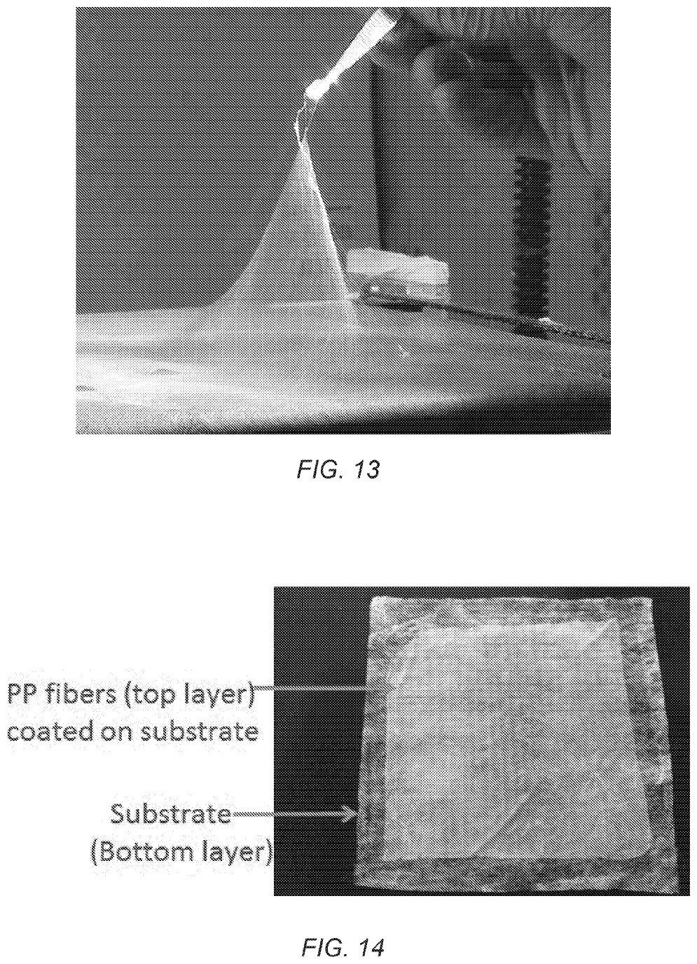

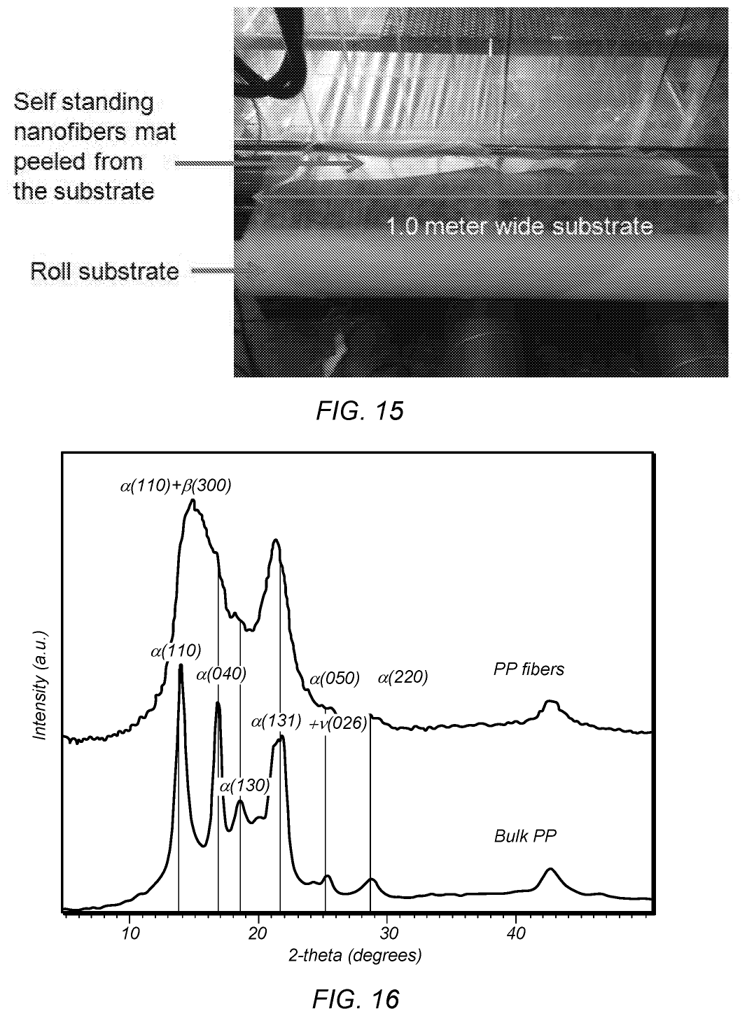

Scanning electron micrographs (FIGS. 11 and 12) show PP fibers of average fiber diameter below 500 nm for MFR of 1550 g/10 min. Fibers were obtained either as free standing nonwoven mats as shown in FIG. 13 (where the nanofiber mat is manually pulled out of the substrate) or deposited on a substrate (FIG. 14). The nanofiber mats were also produced in the continuous processing production equipment. Production equipment has the capabilities to fabricate fibers from polymer solutions (polymer dissolved in solvent(s) at a particular concentration) or polymer melts to coat 1.1 meter wide substrate, where the length of the substrate to be coated with nanofibers mat depends on the length of the roll fed in production equipment. FIG. 15 shows a melt spun polypropylene self-standing continuous nanofibers mat of 50 meter long and 1 meter wide coated on a substrate using the production system. The productivity depends on the number of spinneret heads, spinneret design, number of orifices in the spinneret, flow rate, rotational speed, melt viscosity of polymer material and the fiber diameter requirement of centrifugally spun fibers. The current productivity of melt spun PP varies from 0.04 to 0.08 g/min/orifice. Thus, the number of orifices may be varied to alter the productivity rate. The energy consumption of production system is around 13 kwh/kg of fibers.

PP is a polymorphic material, which has a tendency to crystallize in at least three morphological structures, known as α, β, and γ. These crystal structures have been a subject of intense study. For several years these structures were known to be monoclinic (α), pseudo-hexagonal (β), and triclinic or orthorhombic (γ). The most common monoclinic a structure established in 1960 has been confirmed by subsequent studies except for some adjustments in cell parameters. In the case of the β and γ structures, these remained a puzzle for long time until diligent studies and improve techniques solved the crystal structure puzzle. The γ phase was presented to be an orthorhombic unit cell, while the β phase (distinguishable phase within iPP) was found to be a frustrated trigonal cell with three isochiral lattices. Studies regarding space groups and chain packing geometry of these crystal structures have also contributed to a better understanding of the iPP crystal structure. In the case of nanofibers, the process of structural change differs from unoriented crystallization where nucleation rate, growth rate of the nucleus and the mode of geometrical growth are the parameters that will explain the crystallization kinetics. In oriented crystallization the morphology of the materials becomes strongly dependent on the degree of molecular orientation and the temperature needed to maximize rate of crystallization also increases.

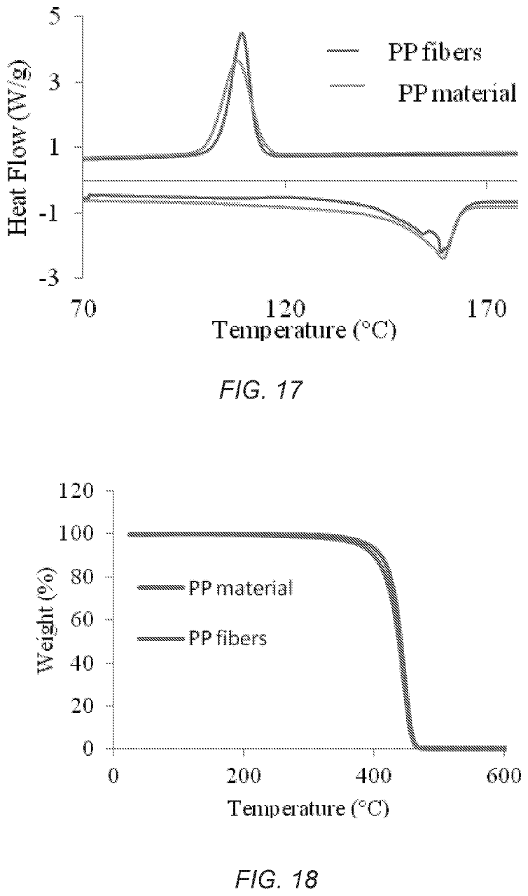

FIG. 16 shows the XRD curves for PP in powder form and developed nanofibers, it can be observed that the powder sample shows distinct peaks usually observed in iPP samples where the monoclinic a structure is predominant as shown by planes (110), (040), (130), (111), (050) and (220). In the nanofiber sample, a broadening of the peaks corresponds to a deformation of the original crystal phases due to the imposed alignment and rapid cooling, a shifting of the α(110) peak towards the β(300) is seen. The β phase (observed under molecular alignment) is observed at 16.2°. As mentioned above, the temperature drop at the exit of the nozzle is significantly high. The nanofibers exit the nozzle and undergo rapid cooling due to the air stream caused by the rotating spinneret. This rapid cooling precludes the extensive formation of crystals as observed also in crystallinity calculations obtained from DSC where the bulk iPP has a crystallinity value of 75% while the developed nanofibers show a crystallinity of 65%. These values were calculated considering an ideal heat of fusion of 188 J/g. Even though the amorphous phase increases, in the DSC plots shown in FIG. 17, a distinct peak in the endothermic peak is observed, this peak represents the oriented meso-phase (β phase) as observed in the XRD spectra. The slight decrease in crystallinity compares well with previous studies conducted on the development of nanofibers. Several studies have shown decreases and others an increase have reported that crystallinity values are affected depending on the glass transition temperature, basically explaining that for polymers with low glass transition temperatures (ductile polymers such as PCL with Tg of −60° C.) an increase in crystallinity is observed in electrospun nanofibers while the opposite is observed for polymers with high transition temperature (rigid polymers).

FIG. 18 shows the TGA curves for both samples. It can be observed that the centrifugal spinning does not cause degradation of the polymer (decrease of molecular weight caused by chain scission). In other nanofiber systems developed from melt spinning, degradation has been observed due to thermal or shear overexposure. The polydispersity of the polymers is usually affected by the long residence time in the system. In the case of fibers fabricated with the lab scale system, the residence time is no more than 8 minutes while the fibers fabricated in the production system underwent a 10 minutes residence time. In both cases degradation was not observed.

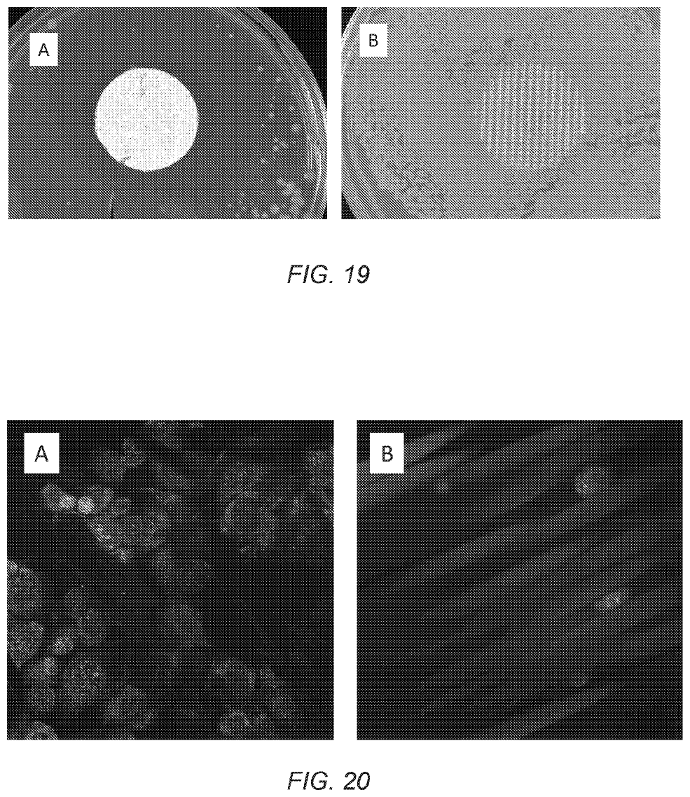

Tests were performed to study the biological properties of PP surgical mesh having melt spun PP fibers that were treated with an antimicrobial agent. FIG. 19 shows the effect of the treated PP fibers on bacteria proliferation. FIG. 19A shows PP surgical mesh that has been coated with antimicrobial treated fibers, while FIG. 19B shows the uncoated PP surgical mesh. As can be seen, the sample coated with the antimicrobial treated fibers shows little bacterial growth, while the untreated surgical mesh has a significant amount of bacteria growing in the media.

Further tests were performed to study the rate of cell proliferation of PP nanofibers versus surgical mesh. FIG. 20A shows PP fibers shows significant cell proliferation, while the surgical mesh shows a lack of cell proliferation.

### Tensile Strength of PP Surgical Mesh

A deterioration of the tensile strength of the mesh or a strained mesh could potentially lead to hernia recurrence or a poor functional result. Hence, materials employed in surgical meshes must possess the minimum mechanical properties necessary to withstand the stresses placed on the abdominal wall. The maximum intra-abdominal pressure generated in a healthy adult, occurs when coughing or jumping and is estimated to be approximately 170 mmHg. Given this information the mesh used to repair abdominal hernias must withstand at least 180 mmHg (20 kPa) before failing.

The tension placed on the abdominal wall can be calculated using Laplace's law relating the tension, pressure, thickness, and diameter of the abdominal wall. According to the thin-walled cylinder model, the total tensile strength is independent of the thickness of the layer. Hence, a physiological tensile strength of 16 N/cm is defined, using a pressure of 20 kPa (2 N/cm² as the maximum pressure to be experienced in the intra-abdominal wall), and 32 cm as the longitudinal diameter of the abdominal wall.

Studies over human abdominal walls have demonstrated that at the maximum tensile strength of 16 N/cm, the abdominal wall in males presents a natural mean distension of 23±7% and 15±5% when tissue is stretched in vertical and horizontal direction, respectively. In females a distension of 32±17% and 17±5% in vertical and horizontal stretching has been observed.

Uniaxial testing of a PP nanofiber mesh was preformed and the results presented below in Table 1. As shown in Table 1, the PP nanofiber mesh has a tensile strength sufficient for use in abdominal wall surgical procedures.

TABLE 1

Direction

| | Longitudinal | Transversal |
|---|---|---|
| Maximum Load (N) | 0.417245 | 0.36987286 |
| Uniaxial Strain (mm/mm) | 9.05475 | 10.716125 |
| Maximum Stress (KPa) | 463.22125 | 435.0725 |

### CONCLUSIONS

The centrifugal spinning process provides a viable alternative for fabricating nanofibers utilizing melt processes. The process has distinct advantages over conventional techniques to fabricate nanofibers. Centrifugal spinning does not require high voltages or specific dielectric properties of the material as required in electrospinning. Centrifugal spinning eliminates the hot and high velocity air required in melt blowing. The polypropylene morphology was analyzed and it is reported that long, continuous nanofibers are obtained from the PP melt with average diameters of sub 500 nm for polypropylene of high MFR of 1550 g/10 min. The process allows for fibers to be collected as free standing nonwoven mats or uniformly deposited in substrates at desired basis weight. An aligned molecular mesophase is formed given the fast cooling of the developed fibers. The crystallinity percentage decreases slightly and thermal degradation was not observed.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. Fibers for medical use comprising polypropylene and an antimicrobial agent.

2. The fibers of claim 1, wherein the fibers have an average diameter of less than about 500 nm.

3. The fibers of claim 1, wherein the polypropylene polymer has a melt flow rate of between about 30 g/10 min to about 1600 g/10 min.

4. The fibers of claim 1, wherein the polypropylene polymer has a glass transition temperature of below about 250° C.

5. The fibers of claim 1, wherein the antimicrobial agent is an antibiotic agent.

6. The fibers of claim 1, wherein the antimicrobial agent is an antifungal agent.

7. The fibers of claim 1, wherein the antimicrobial agent is selected from the group consisting of tannic acid, chitosan, penicillin, streptomycin antibiotics, cefazolin, erythromycin, cefoxitin, cefotetan, and/or fungizone antimycotic.

8. (canceled)

9. A method of producing the polypropylene fibers of any of claims 1-7, comprising:
placing a polypropylene polymer into a body of a fiber producing device, the body comprising one or more openings;
heating the polypropylene polymer to a temperature above the glass transition temperature of the polypropylene polymer;
rotating the fiber producing device at a speed of at least about 500 rpm, wherein rotation of the fiber producing device causes the heated polypropylene polymer in the body to be passed through one or more openings to produce polypropylene microfibers and/or polypropylene nanofibers comprising the polypropylene polymer;
collecting at least a portion of the produced polypropylene microfibers and/or polypropylene nanofibers;
treating the collected polypropylene microfibers and/or polypropylene nanofibers with an antimicrobial agent.

10. The method of claim 9, wherein treating the polypropylene microfibers and/or polypropylene nanofibers comprises dipping the fibers into a solution of the antimicrobial agent.

11. The method of claim 9, wherein treating the polypropylene microfibers and/or polypropylene nanofibers comprises spraying the fibers with a solution of the antimicrobial agent.

12. The method of claim 9, wherein the polypropylene microfibers and/or polypropylene nanofibers are created without subjecting the polypropylene microfibers and/or polypropylene nanofibers, during their creation, to an externally applied electric field.

13. The method of claim 9, wherein the polypropylene microfibers and/or polypropylene nanofibers are collected as a mat of the polypropylene microfibers and/or polypropylene nanofibers.

14. The method of claim 9, wherein the polypropylene microfibers and/or polypropylene nanofibers are collected by depositing the polypropylene microfibers and/or polypropylene nanofibers onto a support.

15. A polypropylene medical mesh comprising polypropylene microfibers and/or polypropylene nanofibers wherein the polypropylene microfibers and/or polypropylene nanofibers comprise an antimicrobial agent, and wherein the polypropylene microfibers and/or polypropylene nanofibers are formed by the method of claim 9.

16. The polypropylene medical mesh of claim 15, further comprising a support coated with the polypropylene microfibers and/or polypropylene nanofibers comprising an antimicrobial agent.

17. The polypropylene medical mesh of claim 15, wherein the polypropylene medical mesh has a biaxial tensile strength of at least about 20 kPa.

18. A method of producing the polypropylene fibers, comprising:
placing a mixture comprising polypropylene polymer and an antimicrobial agent into a body of a fiber producing device, the body comprising one or more openings;
heating the polypropylene polymer to a temperature above the glass transition temperature of the polypropylene polymer;
rotating the fiber producing device at a speed of at least about 500 rpm, wherein rotation of the fiber producing device causes the heated mixture of the polypropylene polymer and the antimicrobial agent in the body to be passed through one or more openings to produce polypropylene microfibers and/or polypropylene nanofibers comprising the polypropylene polymer and the antimicrobial agent;
collecting at least a portion of the produced antimicrobial polypropylene microfibers and/or antimicrobial polypropylene nanofibers.

19. The method of claim 18, wherein the polypropylene microfibers and/or polypropylene nanofibers are created without subjecting the polypropylene microfibers and/or polypropylene nanofibers, during their creation, to an externally applied electric field.

20. The method of claim 18, wherein the polypropylene microfibers and/or polypropylene nanofibers are collected as a mat of the polypropylene microfibers and/or polypropylene nanofibers.

21. The method of claim 18, wherein the polypropylene microfibers and/or polypropylene nanofibers are collected by depositing the polypropylene microfibers and/or polypropylene nanofibers onto a support.

22. A polypropylene medical mesh comprising polypropylene microfibers and/or polypropylene nanofibers wherein the polypropylene microfibers and/or polypropylene nanofibers comprise an antimicrobial agent, and wherein the polypropylene microfibers and/or polypropylene nanofibers are formed by the method of claim 18.

23. The polypropylene medical mesh of claim 18, further comprising a support coated with the polypropylene microfibers and/or polypropylene nanofibers comprising an antimicrobial agent.

24. The polypropylene medical mesh of claim 18, wherein the polypropylene medical mesh has a biaxial tensile strength of at least about 20 kPa.
